(19) United States
(12) Patent Application Publication
(10) Pub. No.: US 2020/0046621 A1
(43) Pub. Date: Feb. 13, 2020

(54) ORAL COMPOSITION

(71) Applicant: KAO CORPORATION, Tokyo (JP)

(72) Inventor: Ryosuke NAGATA, Ichikawa-shi (JP)

(73) Assignee: KAO CORPORATION, Tokyo (JP)

(21) Appl. No.: 16/342,748

(22) PCT Filed: Oct. 17, 2017
(86) PCT No.: PCT/JP2017/037454

§ 371 Date: Apr. 17, 2019

(30) Foreign Application Priority Data
Oct. 18, 2016 (JP) 2016-204434

(51) Int. Cl. A61K 8/55; A61K 8/24; A61K 8/60; A61K 8/73; A61K 8/92; A61Q 11/00

(52) U.S. Cl. CPC A61K 8/55; A61K 8/24; A61Q 11/00; A61K 8/731; A61K 8/92; A61K 8/60

(57) ABSTRACT

The present invention relates to an oral composition which selectively and effectively removes deposited stains present in the interprismatic space on the tooth enamel surface layer and also effectively prevents new stains from adhering and depositing to thereby provide the beautiful gloss that the teeth inherently have. More specifically, the present invention provide an oral composition which comprises the following ingredient (A) phytic acid or a salt thereof and ingredient (B) tripolyphosphoric acid or a salt thereof, wherein a total content of ingredient (A) in terms of phytic acid and ingredient (B) in terms of acid, and a mass ratio of ingredient (B) in terms of acid to ingredient (A) in terms of phytic acid ((B)/(A)) have specific values, the oral composition comprises (C) a specific polyvalent metal or a salt thereof (C) or comprises ingredient (C) in a molar amount of less than 0.1 times that of ingredient (A) in terms of phytic acid, and has a pH at 25° C. of 5.5 or more and 6.5 or less when diluted with water to 30 mass %.

### FIELD OF THE INVENTION

The present invention relates to an oral composition.

### BACKGROUND OF THE INVENTION

Tooth enamel consists of assembled mass of pillar-like substances composed of polycrystalline hydroxyapatite called an enamel rod and, generally at the early stage of life, there are spaces (gaps) between the enamel rods of the enamel, i.e., the interprismatic spaces, which are filled with water and saliva components. Then, an incident light into the enamel scatters due to a large difference in refractive index between the enamel rod and the interprismatic space, thereby making the teeth look white.

However, when substances dissolved in the saliva continue to deposit in the interprismatic space on the tooth enamel surface layer due to dental calculus, plaque, smoking, habitual eating and drinking of coffee or tea, and aging, the interprismatic space is filled, and, consequently the difference in the refractive index between the enamel rod and the interprismatic space becomes smaller to thereby increase the transparency in the enamel, making it easier for an incident light to reach the yellow to brown color dentin located at the deep portion of the enamel, and thus causing the teeth to look yellowish.

Under the circumstance, various developments have been made to enhance the teeth aesthetics. For example, Patent Literature 1 discloses a composition for reforming the interprismatic space containing a specific amount of phytic acid or a salt thereof and having a specific pH value, which selectively removes substances in the interprismatic space caused by aging and the like and reforms the interprismatic space lost, to thereby enhance a teeth whitening effect. Additionally, Patent Literature 2 discloses a dentifrice composition containing a phytic acid compound and particles having a specific average particle size and a collapse strength, which attempts the increase in an effect of removing the teeth stains and a whitening effect.

On the other hand, Patent Literature 3 discloses an oral composition containing specific amounts of phytic acid or a salt thereof and pyrophosphoric acid or a salt thereof in combination under specific conditions such as pH, which removes microstains adhered to the teeth surface, to thereby provide a gloss imparting effect.
(Patent Literature 1) JP-A-2009-263268
(Patent Literature 2) JP-A-2003-335646
(Patent Literature 3) JP-A-2012-219058

### SUMMARY OF THE INVENTION

The present invention relates to an oral composition comprising the following ingredients (A) and (B):

(A) phytic acid or a salt thereof, and

(B) tripolyphosphoric acid or a salt thereof, wherein a total content of ingredient (A) in terms of phytic acid and ingredient (B) in terms of acid is 0.02 mass % or more and 1.4 mass % or less, and a mass ratio of ingredient (B) in terms of acid to ingredient (A) in terms of phytic acid, (B)/(A), is 0.8 or more and 5 or less, and

wherein the oral composition does not comprise (C) one or more polyvalent metals selected from the group consisting of copper, iron, calcium, magnesium, aluminum, zinc, and tin, or a salt thereof, or comprises ingredient (C) in a molar amount of less than 0.1 times that of ingredient (A) in terms of phytic acid, and

a pH at 25° C. when diluted with water to 30 mass % is 5.5 or more and 6.5 or less.

In the arts described in the above Patent Literatures, an effect of removing various stains adhered to the teeth is enhanced to thereby provide a whitening effect, but the study on exerting an effect of preventing adhesion of these various stains is not conducted, thus stains are likely to adhere or deposit to the teeth again due to repeated habitual eating and drinking, aging and the like, and the teeth whitening effect once imparted also tends to be lost in no time. For this reason, a much awaited is the emergence of technology capable of bringing back the inherent healthy color of the teeth and maintaining such a color.

Thus, the present invention relates to an oral composition which selectively and effectively removes deposited stains present in the interprismatic space on the tooth enamel surface layer and also effectively prevents newly deposited stains from adhering to the teeth to thereby provide the beautiful gloss that the teeth inherently have.

Thus, the present inventor conducted various studies and found that an oral composition capable of providing both a deposited stain-selectively removing effect and deposited stain adhesion-preventing effect in the interprismatic space on the tooth enamel surface layer can be obtained when phytic acid or a salt thereof and tripolyphosphoric acid or the like are used in combination in a specific total content and a mass ratio, the content of a specific polyvalent metal or a salt thereof is limited, and a specific pH is ensured.

According to the oral composition of the present invention, deposited stains present in the interprismatic space on the tooth enamel surface layer can be selectively and effectively removed, whereby the beautiful gloss that the teeth inherently have can be imparted. Additionally, new stains are effectively prevented from adhering and depositing to the teeth surface and the interprismatic space and good teeth smoothness can be ensured without causing a friction feeling, whereby smooth gloss of the teeth can be maintained.

### DETAILED DESCRIPTION OF THE INVENTION

Hereinafter, the present invention is described in detail.

Note that the friction feeling means a physically unpleasant feeling at the teeth as if friction is sensed more than necessary when contacting or grinding the teeth each other, and is also a feel which can be sensed when the teeth surface is considerably uneven and consequently loses smoothness, causing roughness.

The oral composition of the present invention contains, as ingredient (A), phytic acid or a salt thereof. Phytic acid is also known as myo-inositol hexaphosphate, a phosphate compound. When such an ingredient (A) is contained, deposited stains present in the interprismatic space can be selectively and effectively removed, to thereby effectively prevent an excessive damage on the enamel, whereby a good gloss can be imparted to the teeth.

Examples of the salt of phytic acid include alkali metal salts such as sodium and potassium and ammonium salts.

The content of ingredient (A) in the oral composition of the present invention in terms of phytic acid is preferably 0.01 mass % or more, more preferably 0.05 mass % or more, further preferably 0.1 mass % or more, further preferably 0.23 mass % or more from the viewpoint of exerting the deposited stain-removing effect well in the interprismatic space. Further, the content of ingredient (A) in the oral composition of the present invention in terms of phytic acid is preferably 1.2 mass % or less, more preferably 1 mass % or less, further preferably 0.7 mass % or less, further preferably 0.5 mass % or less from the viewpoint of suppressing the occurrence of a friction feeling and maintaining the good gloss of the teeth. Additionally, the content of ingredient (A) in the oral composition of the present invention in terms of phytic acid is preferably from 0.01 to 1.2 mass %, more preferably from 0.05 to 1 mass %, further preferably from 0.1 to 0.7 mass %, further preferably from 0.23 to 0.5 mass %.

The oral composition of the present invention contains, as ingredient (B), tripolyphosphoric acid or a salt thereof. When such an ingredient (B) is contained, together with ingredient (A), the teeth surface is coated well to thereby effectively prevent deposited stains from adhering to the teeth surface and the interprismatic space again while ensuring the deposited stain-removing effect in the interprismatic space, whereby the good gloss of the teeth can be maintained.

Examples of the salt of ingredient (B) include alkali metal salts such as sodium and potassium, with sodium being preferable.

The content of ingredient (B) in the oral composition of the present invention in term of acid is preferably 0.01 mass % or more, more preferably 0.1 mass % or more, further preferably 0.3 mass % or more, further preferably 0.4 mass % or more from the viewpoint of exerting the deposited stain adhesion-preventing effect well on the teeth surface and in the interprismatic space. Further, the content of ingredient (B) in the oral composition of the present invention in terms of acid is preferably 1.2 mass % or less, more preferably 1.0 mass % or less, further preferably 0.9 mass % or less, further preferably 0.7 mass % or less from the viewpoint of suppressing the occurrence of a friction feeling and maintaining the good gloss of the teeth. Additionally, the content of ingredient (B) in the oral composition of the present invention in terms of acid is preferably from 0.01 to 1.2 mass %, more preferably from 0.1 to 1.0 mass %, further preferably from 0.3 to 0.9 mass %, further preferably 0.4 from to 0.7 mass %.

In the oral composition of the present invention, the total content of ingredient (A) in terms Of phytic acid and ingredient (B) in terms of acid, (A)+(B), is 0.02 mass or more and 1.4 mass % or less. This range enables the effective provision of both a good stain-removing effect in the interprismatic space and a good deposited stain adhesion-preventing effect on the teeth surface and in the interprismatic space, thereby maintaining the inherent smooth gloss on the teeth while suppressing the occurrence of a friction feeling. Such a total content of ingredient (A) in terms of phytic acid and ingredient (B) in terms of acid, (A)+(B), in the oral composition of the present invention is 0.02 mass % or more, preferably 0.2 mass % or more, more preferably 0.4 mass % or more, further preferably 0.7 mass % or more from the viewpoint of effectively exerting the stain-removing effect in the interprismatic space and the deposited stain adhesion-preventing effect on the teeth surface and in the interprismatic space. Further, the total content of ingredient (A) in terms of phytic acid and ingredient (B) in terms of acid, (A)+(B), in the oral composition of the present invention is 1.4 mass % or less, preferably 1.3 mass % or less, more preferably 1.2 mass % or less, further preferably 0.9 mass % or less from the viewpoint of effectively preventing dissolution of the tooth enamel and the occurrence of a friction feeling and effectively maintaining the smooth gloss of the teeth. Additionally, the total content of ingredient (A) in terms of phytic acid and ingredient (B) in terms of acid ((A)+(B)) in the oral composition of the present invention is 0.02 mass % or more and 1.4 mass % or less, preferably from 0.2 to 1.3 mass %, more preferably from 0.4 to 1.2 mass %, further preferably from 0.7 to 0.9 mass %.

The mass ratio of ingredient (B) in terms of acid to ingredient (A) in terms of phytic acid, (B)/(A), is 0.8 or more, preferably 1 or more, more preferably 1.2 or more, further preferably 1.5 or more from the viewpoint of effectively providing both a good stain-removing effect in the interprismatic space and a good deposited stain adhesion-preventing effect on the teeth surface and in the interprismatic space. Further, the mass ratio of ingredient (B) in terms of acid to ingredient (A) in terms of phytic acid, (B)/(A) is 5 or less, preferably 4 or less, more preferably 3 or less, further preferably 2 or less from the viewpoint of effectively preventing dissolution of the tooth enamel and the occurrence of a friction feeling and effectively maintaining the smooth gloss of the teeth. Additionally, the mass ratio of ingredient (B) in terms of acid to ingredient (A) in terms of phytic acid, (B)/(A), is 0.8 or more and 5 or less, preferably from 1 to 4, more preferably from 1.2 to 3, further preferably from 1.5 to 2.

In the oral composition of the present invention, there is a limitation of containing (C) one or more polyvalent metals selected from the group consisting of copper, iron, calcium, magnesium, aluminum, zinc, and tin, or a salt thereof. Such an ingredient (C) may make ingredient (A) insoluble and reduce the good stain-removing effect in the interprismatic space, due to which the containing is limited for also effectively ensuring the smooth gloss of the teeth and the durability thereof. The oral composition of the present invention contains ingredient (C) in a molar amount of less than 0.1 times, preferably 0.02 times or less that of ingredient (A) in terms of phytic acid, or does not contain ingredient (C).

Note that the content of such an ingredient (C) is the value measured by ICP optical emission spectrometry (ICP optical emission spectrometer: Perkin Elmer Optima 5300DV) in terms of the sum of polyvalent cations of ingredient (C).

The oral composition of the present invention, when containing ingredient (A) and ingredient (B), preferably contains (D) xylitol from the viewpoint of more effectively reducing a friction feeling. The content of such an ingredient (D) in the oral composition of the present invention is preferably 1 mass % or more, more preferably 2 mass % or more, further preferably 3 mass % or more from the viewpoint of effectively reducing a friction feeling of ingredient (A) and ingredient (B), Further, the content of ingredient (D) in the oral composition of the present invention is preferably 15 mass % or less, more preferably 12 mass % or less, and further preferably 8 mass % or less from the viewpoint of effectively reducing a friction feeling of the composition, effectively providing a suitable viscidity of the composition, and both a good stain-removing effect in the interprismatic space and a good deposited stain adhesion-preventing effect on the teeth surface and in the interprismatic space. Additionally, the content of ingredient (D) in the oral composition of the present invention is preferably from 1 to 15 mass %, more preferably from 2 to 12 mass %, further preferably from 3 to 8 mass %.

The mass ratio of the content of ingredient (B) to the content of ingredient (D), (B)/(D), is preferably 0.01 or more, more preferably 0.03 or more, further preferably 0.06 or more from the viewpoint of providing both a good stain-removing effect in the interprismatic space and a good deposited stain adhesion-preventing effect on the teeth surface and in the interprismatic space. Further, the mass ratio of the content of ingredient (B) to the content of ingredient (D), (B)/(D), is preferably 0.4 or less, more preferably 0.3 or less, further preferably 0.18 or less, further preferably 0.14 or less from the viewpoint of both effectively reducing a friction feeling of the composition and effectively providing a suitable viscidity of the composition. Additionally, the mass ratio of the content of ingredient (B) to the content of ingredient (D), (B)/(D), is preferably from 0.01 to 0.4, more preferably from 0.03 to 0.3, further preferably from 0.03 to 0.18, further preferably from 0.06 to 0.14.

The mass ratio of the total content of ingredient (A) in terms of phytic acid and ingredient (B) in terms of acid to the content of ingredient (D), {(A)+(B)}/(D), is preferably 0.02 or more, more preferably 0.08 or more, further preferably 0.1 or more, further preferably 0.14 or more from the viewpoint of effectively providing both a good stain-removing effect in the interprismatic space and a good deposited stain adhesion-preventing effect on the teeth surface and in the interprismatic space. Further, the mass ratio of the total content of ingredient (A) in terms of phytic acid and ingredient (B) in terms of acid to the content of ingredient (D), {(A)+(B)}/(D), is preferably 0.35 or less, more preferably 0.3 or less, further preferably 0.24 or less, further preferably 0.2 or less from the viewpoint of both effectively reducing a friction feeling of the composition and effectively providing a suitable viscidity of the composition. Additionally, the mass ratio of the total content of ingredient (A) in terms of phytic acid and ingredient (B) in terms of acid to the content of ingredient (D), {(A)+(B)}/(D), is preferably from 0.02 to 0.35, more preferably from 0.08 to 0.3, further preferably from 0.1 to 0.24, further preferably from 0.14 to 0.2.

The oral composition of the present invention preferably contains (E) monofluorophosphoric acid or an alkali metal salt thereof from the viewpoint of, while increasing an amount of fluorine adsorbed to the teeth, more effectively reducing a friction feeling when ingredient (A) and ingredient (B) are used in combination.

Examples of the alkali metal of alkali metal salt of ingredient (E) include sodium and potassium, with sodium being preferable.

The content of ingredient (E) in the oral composition of the present invention in terms of the fluorine atom is preferably 100 ppm or more, more preferably 500 ppm or more, further preferably 800 ppm or more from the viewpoint of adsorbing fluorine to the teeth well and the viewpoint of effectively reducing a friction feeling. Further, the content of ingredient (E) in the oral composition of the present invention in terms of the fluorine atom is preferably 2,000 ppm or less, more preferably 1,500 ppm or less, and further preferably 1,100 ppm or less from the viewpoint of adsorbing fluorine to the teeth well and the viewpoint of safety. Additionally, the content of ingredient (E) in the oral composition of the present invention in terms of the fluorine atom is preferably from 100 to 2,000 ppm, more preferably from 500 to 1,500 ppm, further preferably from 800 to 1,100 ppm.

The content of ingredient (E) in the oral composition of the present invention is preferably 0.08 mass % or more, more preferably 0.4 mass % or more, further preferably 0.6 mass % or more from the viewpoint of adsorbing fluorine to the teeth well and the viewpoint of effectively reducing a friction feeling. Further, the content of ingredient (E) in the oral composition of the present invention is preferably 1.5 mass % or less, more preferably 1.2 mass % or less, further preferably 0.85 mass % or less from the viewpoint of adsorbing fluorine to the teeth well and the view point of safety. Additionally, the content of ingredient (E) in the oral composition of the present invention is preferably from 0.08 to 1.5 mass %, more preferably from 0.4 to 1.2 mass %, further preferably from 0.6 to 0.85 mass %.

The mass ratio of ingredient (B) in teLLlts of acid to the content of ingredient (E), (B)/(E), is preferably 0.1 or more, more preferably 0.3 or more, and further preferably 0.5 or more from the viewpoint of adsorbing fluorine to the teeth well and effectively reducing a friction feeling. Further, the mass ratio of ingredient (B) in terms of acid to the content of ingredient (E), (B)/(E), is preferably 1.5 or less, more preferably 1.3 or less, further preferably 1 or less from the viewpoint of adsorbing fluorine to the teeth well and the viewpoint of safety,. Additionally, the mass ratio of ingredient (B) in terms of acid to the content of ingredient (E), (B)/(E), is preferably from 0.1 to 1.5, more preferably from 0.3 to 1.3, further preferably from 0.5 to 1.

The mass ratio of the total content of ingredient (A) in terms of phytic acid and ingredient (B) in terms of acid to the content of ingredient (E), {(A)+(B)}/(E), is preferably 0.2 or more, more preferably 0.4 or more, further preferably 0.6 or more, further preferably 0.9 or more from the viewpoint of adsorbing fluorine to the teeth well and effectively reducing a friction feeling when using ingredient (A) and ingredient (B) combination. Further, the mass ratio of the total content of ingredient (A) in terms of phytic acid and ingredient (B) in terms of acid to the content of ingredient (E) ({(A)+(B)}/(E)) is preferably 3 or less, more preferably 2 or less, further preferably 1.7 or less, further preferably 1.4 or less from the viewpoint of adsorbing fluorine to the teeth well and the viewpoint of safety. Additionally, the mass ratio of the total content of ingredient (A) in terms of phytic acid and ingredient (B) in terms of acid to the content of ingredient (E), {(A)+(B)}/(E), is preferably from 0.2 to 3, more preferably from 0.4 to 2, further preferably from 0.6 to 1.7, furthermore preferably from 0.9 to 1.4.

The oral composition of the present invention, when in a form of a dentifrice composition such as toothpaste or tooth powder, preferably contains (F) a binder containing (f) a cellulose binder from the viewpoint of retaining and spreading the composition close to the teeth surface while having a suitable viscosity to effectively and efficiently exert the stain-removing effect in the interprismatic space and the deposited stain adhesion-preventing effect on the teeth surface and in the interprismatic space and the viewpoint of maintaining the smooth gloss of the teeth. Examples of the cellulose binder (f) contained in ingredient (F) specifically include one or more selected from the group consisting of carboxy methyl cellulose or a salt thereof and hydroxyalkyl celluloses such as hydroxyethyl cellulose and hydroxypropyl cellulose, with carboxy methyl cellulose or a salt thereof being preferable.

Such a carboxy methyl cellulose or a salt thereof is preferably a carboxy methyl cellulose having an etherification degree of 0.95 to 1.5, or a salt thereof. The etherification degree of the carboxy methyl cellulose or a salt thereof is preferably 0.95 or more, more preferably 1.0 or more; and preferably 1.5 or less, more preferably 1.15 or less from the viewpoint of imparting a suitable viscosity to retain or spread the composition close to the teeth surface. Further, the etherification degree of the carboxy methyl cellulose or a salt thereof is preferably from 0.95 to 1.5, more preferably from 1.0 to 1.15 from the viewpoint of imparting a suitable viscosity to retain and spread the composition close to the teeth surface.

The content of carboxy methyl cellulose having an etherification degree of 0.95 to 1.5 or a salt thereof in the oral composition is preferably 0.1 mass % or more, more preferably 0.3 mass % or more, further preferably 0.5 mass % or more; and preferably 1.4 mass % or less, more preferably 1.2 mass % or less, further preferably 0.9 mass % or less from the viewpoint of retaining and spreading the composition close to the teeth surface while having a suitable viscosity to effectively and efficiently exert the stain-removing effect in the interprismatic space and the deposited stain adhesion-preventing effect on the teeth surface and in the interprismatic space. Further, the content of carboxy methyl cellulose having an etherification degree of 0.95 to 1.5 or a salt thereof in the oral composition of the present invention is preferably from 0.1 to 1.4 mass %, more preferably from 0.3 to 1.2 mass %, further preferably from 0.5 to 0.9 mass %.

Note that the etherification degree of carboxy methyl cellulose or a salt thereof refers to the substitution degree of a carboxymethyl group per glucose unit. The etherification degree can be determined in accordance with, for example, the CMC Kogyokai analysis method (ashing method). One g of carboxy methyl cellulose or a salt thereof is precisely weighed, placed in a porcelain crucible and subjected to ashing at 600° C., then the sodium oxide produced by ashing is titrated with N/10 sulfuric acid using phenolphthalein as an indicator, whereby the titer Y mL per gram of carboxy methyl cellulose or a salt thereof is inserted in the following formula for calculation, to thereby determine an etherification degree.

Etherification degree=(162×Y)/(10,000-80×Y)

Examples of the salt of carboxy methyl cellulose include alkali metal salts such as sodium and potassium, and ammonium salts, with sodium carboxy methyl cellulose being preferable.

The content of cellulose binder (f) in the oral composition of the present invention is preferably 0.2 mass % or more, more preferably 0.4 mass % or more, further preferably 0.6 mass % or more; and preferably 2 mass % or less, more preferably 1.8 mass % or less, further preferably 1.6 mass % or less from the viewpoint of retaining and spreading the composition close to the teeth surface while having a suitable viscosity, to effectively and efficiently exert the stain-removing effect in the interprismatic space and the deposited stain adhesion-preventing effect on the teeth surface and in the interprismatic space. Further, the content of cellulose binder (f) in the oral composition of the present invention is preferably from 0.2 to 2 mass %, more preferably from 0.4 to 1.8 mass %, further preferably from 0.6 to 1.6 mass %.

Further, the oral composition of the present invention may contain a binder other than the above cellulose binder (f) as the above binder (F). Examples of the binder (F) other than the cellulose binder (f) include one or more selected from the group consisting of sodium alginate, carrageenan, xanthan gum, sodium polyacrylate, pectin, gum tragacanth, gum arabic, guar gum, karaya gum, locust bean gum, gellan gum, tamarind gum, psyllium seed gum, polyvinyl alcohol, sodium chondroitin sulfate, and methoxyethylene-maleic anhydride copolymer. The content of ingredient (F) other than the cellulose binder (f) in the oral composition of the present invention is preferably 0.01 mass % or more, more preferably 0.05 mass % or more, further preferably 0.1 mass % or more. Further, the content of ingredient (F) other than the cellulose binder (f) in the oral composition of the present invention is preferably 1 mass % or less, more preferably 0.8 mass % or less, further preferably 0.6 mass % or less.

The oral composition of the present invention, when in a form of a dentifrice composition such as toothpaste or tooth powder, preferably further contains (G) thickening silica having an oil absorption of 200 mL/100 g or more and 400 mL/100 g or less together with ingredient (F) from the viewpoint of retaining and spreading the composition close to the teeth surface while having a suitable viscosity to effectively and efficiently exert the stain-removing effect in the interprismatic space and the deposited stain adhesion-preventing effect on the teeth surface and in the interprismatic space. More specifically, the thickening silica of ingredient (G) is a silica having a high oil absorption and different from the silica used as an abrasive. The oil absorption herein indicates an amount of the oil that silica can support and means the value specified by the amount of boiled linseed oil absorbed when measured by the measurement method of JIS K5101-13-2 (established in 2004). For the thickening silica, commercial products can be used such as Sylysia and Sylopure (Fuji Silysia Chemical Ltd), Tixosil (Rhodia), Sorbosil (Ineos Silica), Finesil (Tokuyama Corporation), and NIPGEL (Tosoh Silica Corporation). The oil absorption of ingredient (G) is preferably from 220 to 380 mL/100 g, more preferably from 230 to 350 mL/100 g.

The content of ingredient (G) in the oral composition of the present invention is preferably 1 mass % or more, more preferably 3 mass % or more, further preferably 5 mass % or more; and preferably 15 mass % or less, more preferably 12 mass % or less, further preferably 9 mass % or less from the viewpoint of imparting a suitable viscosity to the composition together with ingredient (F) and the viewpoint of retaining and spreading the composition close to the teeth surface to effectively exert the stain-removing effect in the interprismatic space and the deposited stain adhesion-preventing effect on the teeth surface and in the interprismatic space. Further, the content of ingredient (G) in the dentifrice composition of the present invention in the oral composition of the present invention is preferably 1 mass % or more and 15 mass % or less, more preferably from 3 to 12 mass %, further preferably from 5 to 9 mass %.

The oral composition of the present invention, when in a form of a dentifrice composition such as toothpaste or tooth powder, preferably further contains granules containing a water-insoluble inorganic binder from the viewpoint of enhancing the deposited stain-removing effect not only in the interprismatic space but also on the teeth surface, assisting the deposited strain adhesion-suppressing effect due to permeation of ingredient (A) and ingredient (B) to the interprismatic space and adsorption of ingredient (A) and ingredient (B) to the teeth surface, and effectively maintaining good gloss of the teeth. Examples of the water-insoluble inorganic binder include, in addition to silicon compounds such as colloidal silica, magnesium aluminometasilicate, synthetic aluminum silicate, and calcium silicate, one or more selected from the group consisting of bentonite, montmorillonite, kaolin, aluminum hydroxide gel, alumina sol, and synthetic hydrotalcite, with silicon compounds being preferable, colloidal silica, magnesium aluminometasilicate, and synthetic aluminum silicate being more preferable, and colloidal silica being further preferable.

The content of water-insoluble inorganic binder in the granules is preferably from 10 to 45 mass %, and more preferably from 10 to 40 mass %.

The granules may contain, for example, an abrasive powder other than the water-insoluble inorganic binder such as calcium carbonate, anhydrous silicic acid, aluminium hydroxide, and zeolite, and preferably contains anhydrous silicic acid. The content of abrasive powder in the granules is preferably from 40 to 90 mass %, more preferably from 50 to 85 mass %. Further, the granules can contain a water-insoluble fiber, and examples of the water-insoluble fiber include cellulose, water-insoluble hemicellulose, lignin, chitin, and chitosan, with cellulose being preferable. The content of water-insoluble fiber in the granules is preferably from 1 to 40 mass %, more preferably from 2 to 30 mass %.

The average particle size of granules is preferably from 50 to 500 μm, more preferably from 75 to 300 μm, from the viewpoint of suppressing an increase in a feeling of foreign matter derived from the granules in the oral cavity and the viewpoint of further enhancing the deposited stain-removing effect of the oral composition of the present invention in the interprismatic space and on the teeth surface. The average particle size of granules herein refers to the value measured by the sieving method (sonic sieve, Tsutsui Scientific Instruments, Co., Ltd. SW-20-AT).

The content of granules containing the above water-insoluble inorganic binder in the oral composition of the present invention is preferably 1 mass % or more, more preferably 3 mass % or more; and preferably 10 mass % or less, more preferably 8 mass % or less, from the viewpoint of effectively enhancing the deposited stain-removing effect in the interprismatic space and on the teeth surface without excessively increasing a feeling of foreign matter derived from the granules in the oral cavity. The content of granules containing the above water-insoluble inorganic binder in the oral composition of the present invention is preferably from 1 to 10% by mass, more preferably form 3 to 8% by mass.

The oral composition of the present invention, when in a form of a dentifrice composition such as toothpaste or tooth powder, can further contain an abrasive from the viewpoint of effectively enhancing the deposited stain-removing effect not only in the interprismatic space but also on the teeth surface. Examples of the abrasive include calcium phosphate, calcium hydrogen phosphate, calcium carbonate, aluminium hydroxide, aluminum silicate, zirconium silicate, and abrasive silica (an oil absorption measured by the method in accordance with JIS K5101-13-2 is 50 to 150 mL/100 g). The abrasive generally used is one having an RDA value (Radioactive Dentine Abrasion values, the value measured by ISO11609 Abradability Test Method Appendix A) of 20 to 250. The content of abrasive in the oral composition of the present invention is preferably 1 mass % or more; and preferably 20 mass % or less, more preferably 15 mass % or less.

The oral composition of the present invention preferably contains one or more anionic surfactants selected from the group consisting of alkyl sulfates and acyl methyl taurine salts from the viewpoint of retaining and spreading the composition close to the teeth surface while having a suitable viscidity or viscosity to effectively and efficiently exert the deposited stain-removing effect in the interprismatic space and the deposited stain adhesion-preventing effect on the teeth surface and in the interprismatic space. Examples of the alkyl sulfate include sodium lauryl sulfate and sodium myristyl sulfate, and examples of the acyl methyl taurine salt include sodium salts and potassium salts of, for example, lauroyl methyl taurate, myristoyl methyl taurate, palmitoyl methyl taurate, and stearoyl methyl taurate. Of these, sodium lauryl sulfate and sodium lauroyl methyl taurate are preferable from the viewpoint of imparting a suitable viscidity or viscosity.

When the oral composition of the present invention is in a form of a liquid oral composition such as a mouthwash or a liquid dentifrice, the content of one or more anionic surfactants selected from the group consisting of alkyl sulfates and acyl methyl taurine salts in the oral composition of the present invention is preferably from 0.5 to 5 mass %, more preferably from 1 to 3 mass %. Further, when the oral composition of the present invention is in a form of a dentifrice composition such as toothpaste or tooth powder, the content of one or more anionic surfactants selected from the group consisting of alkyl sulfates and acyl methyl taurine salts in the oral composition of the present invention is preferably from 0.5 to 5 mass %, more preferably from 1 to 3 mass %.

The oral composition of the present invention contains (H) water. The term “water” of ingredient (H) in the present invention refers to the total water contained in the oral composition containing not only purified water blended to the oral composition but also water contained in each of the blended ingredients, such as a 70% sorbitol liquid (aqueous solution) used in formulating prescribing. When such a water of ingredient (H) is contained, each ingredient can be dispersed or dissolved well and spread well in the oral cavity while ensuring a good shape-retaining property to exert the good deposited stain-removing effect in the interprismatic space and a the high deposited stain adhesion-preventing effect on the teeth surface and in the interprismatic space.

For example, when the oral composition of the present invention is in the form of a liquid oral composition such as a mouthwash or a liquid dentifrice, the content of ingredient (H) in the oral composition of the present invention is preferably 50 mass % or more, more preferably 70 mass % or more, further preferably 80 mass % or more; and preferably 99 mass % or less, more preferably 95 mass % or less, further preferably less than 90 mass %. Further, when the oral composition of the present invention is a liquid oral composition such as a mouthwash or a liquid dentifrice, the content of ingredient (H) in the oral composition of the present invention is preferably form 50 to 99 mass %, more preferably from 70 to 95 mass %, further preferably 80 mass or more and less than 90 mass %.

Furthermore, when the oral composition of the present invention is in a form of a dentifrice composition such as toothpaste or tooth powder, the content of ingredient (H) in the oral composition of the present invention is preferably 10 mass % or more, more preferably 12 mass % or more; and preferably 60 mass % or less, more preferably 50 mass % or less. Further, when the oral composition of the present invention is in a form of a dentifrice composition such as toothpaste or tooth powder, the content of ingredient (H) is preferably from 10 to 60 mass %, more preferably from 12 mass % or more and 50 mass % or less.

When the oral composition of the present invention is in a form of a dentifrice composition such as toothpaste or tooth powder, the water content thereof can be calculated from the content of the blended water and the content of water in the blended ingredients, and the content can also be measured with, for example, a Karl Fischer moisture meter. As the Karl Fischer moisture meter, for example, a trace moisture measuring device (Hiranuma Sangyo Co., Ltd.) can be used. In this device, 5 g of the dentifrice composition is taken and suspended in 25 g of anhydrous methanol, and the water content in 0.02 g of this suspension can be measured.

The oral composition of the present invention may contain, in addition to the above-mentioned ingredients, a sugar alcohol such as sorbitol, erythritol, reduced palatinose, or mannitol; a humectant such as glycerin, polyethyleneglycol, or propyleneglycol; a pH adjusting agent; a surfactant other than the above-mentioned anionic surfactant; a fluoride; a bactericide; a preservative; a flavoring agent; and a dye, within a range not impairing the advantageous effects of the present invention.

The oral composition of the present invention has pH at 25° C. of 5.5 or more, preferably 5.8 or more; and 6.5 or less, preferably 6.2 or less, when diluted with water to 30 mass % from the viewpoint of effectively and efficiently exerting the deposited stain-removing effect in the interprismatic space and the deposited stain adhesion-preventing effect on the teeth surface and in the interprismatic space and imparting the beautiful gloss that the teeth inherently have.

The pH of the oral composition of the present invention cannot be precisely measured in a case of, for example, an oral composition having a high viscosity such as toothpaste, and for this reason, the pH of the oral composition of the present invention is defined by using the composition diluted with water to 30 mass %. The composition diluted with water to 30 mass % is used because such a condition is assumed that the oral composition is applied to the oral cavity and diluted with saliva and the like. Note that water is purified water, and distilled water or ion exchange water is used.

The viscosity at 25° C. of the oral composition of the present invention is preferably 1,500 dPa·s or more, more preferably 1,800 dPa·s or more ;and preferably 3,500 dPa·s or less, more preferably 3,000 dPa·s or less, from the viewpoint of retaining and spreading the composition close to the teeth surface while having a suitable viscidity or viscosity to effectively and efficiently exert the deposited stain-removing effect in the interprismatic space and the deposited stain adhesion-preventing effect on the teeth surface and in the interprismatic space. Further, the viscosity at 25° C. of the oral composition of the present invention is preferably from 1,500 to 3,500 dPa·s, more preferably from 1,800 to 3,000 dPa·s.

The viscosity herein can be measured using a Helipath viscometer (VISCOMETER TVB-10, Toki Sangyo Co., Ltd.) at a measurement temperature of 25° C. under the measurement conditions of a rotor T-C at a rotation speed of 2.5 r/min for 1 minute.

Examples of the form of the oral composition of the present invention include liquid oral compositions such as mouthwash and liquid dentifrice and dentifrice compositions such as toothpaste and tooth powder, but dentifrice compositions such as toothpaste and tooth powder are preferable from the viewpoint of effectively exerting the deposited stain-removing effect in the interprismatic space and the deposited stain adhesion-preventing effect on the teeth surface and in the interprismatic space and continuously imparting the beautiful gloss to the teeth.

From the viewpoint of effectively exerting the deposited stain-removing effect in the interprismatic space and the deposited stain adhesion-preventing effect on the teeth surface and in the interprismatic space and continuously imparting beautiful gloss to the teeth, the oral composition of the present invention is most preferably a toothpaste comprising:

(A) 0.23 to 0.5 mass % of phytic acid or a salt thereof and

(B) 0.4 to 1.2 mass % of tripolyphosphoric acid or a salt thereof, wherein a total content of ingredient (A) in terms of phytic acid and ingredient (B) in terms of acid is 0.2 mass % or more and 1.4 mass % or less, and a mass ratio of ingredient (B) in terms of acid to ingredient (A) in terms of phytic acid, (B)/(A), is 0.8 or more and 4 or less, and the toothpaste does not comprise (C) one or more polyvalent metals selected from the group consisting of copper, iron, calcium, magnesium, aluminum, zinc, and tin, or a salt thereof, or comprises ingredient (C) in a molar amount of less than 0.1 times that of ingredient (A) in terms of phytic acid, the toothpaste comprising:

(D) 3 to 13 mass % of xylitol, and

(F) 0.6 to 1.6 mass % of carboxy methyl cellulose having an etherification degree of 0.95 to 1.5 or a salt thereof, and wherein the toothpaste has a pH at 25° C. of 5.5 or more and 6.5 or less when diluted with water to 30 mass %.

With respect to the above-described embodiments, the present invention further discloses the following oral compositions.

[1] An oral composition comprising the following ingredients (A) and (B):

(A) phytic acid or a salt thereof, and

(B) tripolyphosphoric acid or a salt thereof, wherein a total content of ingredient (A) in terms of phytic acid and ingredient (B) in terms of acid is 0.02 mass % or more and 1.4 mass % or less, and a mass ratio of ingredient (B) in terms of acid to ingredient (A) in terms of phytic acid, (B)/(A), is 0.8 or more and 5 or less,

the oral composition does not comprise (C) one or more polyvalent metals selected from the group consisting of copper, iron, calcium, magnesium, aluminum, zinc, and tin, or a salt thereof, or comprises ingredient (C) in a molar amount of less than 0.1 times that of ingredient (A) in terms of phytic acid is comprised, and

the oral composition has a pH at 25° C. of 5.5 or more and 6.5 or less when diluted with water to 30 mass %.

[2] The oral composition according to aspect [1], wherein the total content of ingredient (A) in terms of phytic acid and ingredient (B) in terms of acid, (A)+(B), is preferably 0.2 mass % or more, more preferably 0.4 mass % or more, further preferably 0.7 mass % or more; and preferably 1.3 mass % or less, more preferably 1.2 mass % or less, further preferably 0.9 mass % or less.

[3] The oral composition according to aspect [1] or [2], wherein the mass ratio of ingredient (B) in terms of acid to ingredient (A) in terms of phytic acid, (B)/(A), is preferably 1 or more, more preferably 1.2 or more, further preferably 1.5 or more; and preferably 4 or less, more preferably 3 or less, further preferably 2 or less.

[4] The oral composition according to any one of aspects [1] to [3], wherein a content of ingredient (A) in terms of phytic acid is preferably 0.01 mass % or more, more preferably 0.05 mass % or more, further preferably 0.1 mass % or more, further preferably 0.23 mass % or more; and preferably 1.2 mass % or less, more preferably 1 mass % or less, further preferably 0.7 mass. or less, further preferably 0.5 mass % or less.

[5] The oral composition according to any one of aspects [1] to [4], wherein a content of ingredient (B) in terms of acid is preferably 0.01 mass % or more, more preferably 0.1 mass % or more, further preferably 0.3 mass % or more, further preferably 0.4 mass % or more; and preferably 1.2 mass % or less, more preferably 1.0 mass % or less, further preferably 0.9 mass % or less, further preferably 0.7 mass % or less.

[6] The oral composition according to any one of aspects [1] to [5], wherein the oral composition preferably comprises ingredient (C) in a molar amount of 0.02 times or less that of ingredient (A) in terms of phytic acid, or does not comprise ingredient (C).

[7] The oral composition according to any one of aspects [1] to [6], wherein the oral composition preferably comprises (D) xylitol, wherein a content of ingredient (D) is preferably 1 mass % or more, more preferably 2 mass % or more, further preferably 3 mass % or more; and preferably 15 mass % or less, more preferably 12 mass % or less, further preferably 8 mass % or less.

[8] The oral composition according to aspect [7], wherein a mass ratio of the content of ingredient (B) to the content of ingredient (D), (B)/(D), is preferably 0.01 or more, more preferably 0.03 or more, further preferably 0.06 or more; and preferably 0.4 or less, more preferably 0.3 or less, further preferably 0.18 or less, further preferably 0.14 or less.

[9] The oral composition according to aspect [7] or [8], wherein a mass ratio of the total content of ingredient (A) in terms of phytic acid and ingredient (B) in terms of acid to the content of ingredient (D), {(A)+(B)}/(D), is preferably 0.02 or more, more preferably 0.08 or more, further preferably 0.1 or more, further preferably 0.14 or more; and preferably 0.35 or less, more preferably 0.3 or less, further preferably 0.24 or less, further preferably 0.2 or less.

[10] The oral composition according to any one of aspects [1] to [9], wherein the oral composition preferably comprises (E) monofluorophosphoric acid or an alkali metal salt thereof, wherein a content of ingredient (E) in terms of the fluorine atom is preferably 100 ppm or more, more preferably 500 ppm or more, further preferably 800 ppm or more; and preferably 2,000 ppm or less, more preferably 1,500 ppm or less, further preferably 1,100 ppm or less.

[11] The oral composition according to aspect [10], wherein a mass ratio of ingredient (B) in terms of acid to the content of ingredient (E), (B)/(E), is preferably 0.1 or more, more preferably 0.3 or more, further preferably 0.5 or more; and preferably 1.5 or less, more preferably 1.3 or less, further preferably 1 or less.

[12] The oral composition according to aspect [10] or [11], wherein a mass ratio of the total content of ingredient (A) in terms of phytic acid and ingredient (B) in terms of acid to the content of ingredient (E), {(A)+(B)}/(E), is preferably 0.2 or more, more preferably 0.4 or more, further preferably 0.6 or more, further preferably 0.9 or more; and preferably 3 or less, more preferably 2 or less, further preferably 1.7 or less, further preferably 1.4 or less.

[13] The oral composition according to any one of aspects [1] to [12], wherein the oral composition preferably comprises (F) a binder containing (f) a cellulose binder, wherein examples of ingredient (f) contained in ingredient (F) includes preferably one or more selected from the group consisting of carboxy methyl cellulose or a salt thereof and hydroxyalkyl celluloses such as hydroxy ethyl cellulose and hydroxypropyl cellulose, more preferably carboxy methyl cellulose or a salt thereof.

[14] The oral composition according to aspect [13], wherein the content of ingredient (f) is preferably 0.2 mass % or more, more preferably 0.4 mass % or more, further preferably 0.6 mass % or more; and preferably 2 mass % or less, more preferably 1.8 mass % or less, further preferably 1.6 mass % or less.

[15] The oral composition according to aspect [13] or [14], wherein the content of ingredient (F) other than ingredient (t) is preferably 0.01 mass % or more, more preferably 0.05 mass % or more, further preferably 0.1 mass % or more; and preferably 1 mass % or less, more preferably 0.8 mass % or less, further preferably 0.6 mass % or less.

[16] The oral composition according to any one of aspects [13] to [15], wherein the oral composition preferably comprises carboxy methyl cellulose or a salt thereof as the cellulose binder (f), wherein an etherification degree of carboxy methyl cellulose or a salt thereof is preferably 0.95 or more, more preferably 1.0 or more; and preferably 1.5 or less, more preferably 1.15 or less.

[17] The oral composition according to any one of aspects [1] to [16], wherein the oral composition preferably further comprises (G) thickening silica having an oil absorption of 200 mL/100 g or more and 400 mL/100 g or less together with ingredient (F), wherein a content of ingredient (G) is preferably 1 mass % or more, more preferably 3 mass % or more, further preferably 5 mass % or more; and preferably 15 mass % or less, more preferably 12 mass % or less, further preferably 9 mass % or less.

[18] The oral composition according to any one of aspects [1] to [17], wherein the oral composition preferably comprises granules containing a water-insoluble inorganic binder, wherein the water-insoluble inorganic binder is preferably one or more selected from the group consisting of colloidal silica, magnesium aluminometasilicate, synthetic aluminum silicate, calcium silicate, bentonite, montmorillonite, kaolin, aluminium hydroxide gel, alumina sol, and synthetic hydrotalcite, with colloidal silica, magnesium aluminometasilicate, synthetic aluminum silicate being more preferable, and colloidal silica being further preferable.

[19] The oral composition according to aspect [18], wherein a content of the water-insoluble inorganic binder in the granules is preferably from 10 to 45 mass %, more preferably from 10 to 40 mass %.

[20] The oral composition according to aspect [18] or [19], wherein a content of the granules containing the water-insoluble inorganic binder is preferably 1 mass % or more, more preferably 3 mass % or more; and preferably 10 mass % or less, more preferably 8 mass % or less.

[21] The oral composition according to any one of aspects [1] to [20], wherein, in the case of the oral composition of the present invention being a liquid oral composition, a content of one or more anionic surfactants selected from the group consisting of alkyl sulfates and acyl methyl taurine salts is preferably from 0.5 to 5 mass %, more preferably from 1 to 3 mass %, and in the case of the oral composition of the present invention being a dentifrice composition, a content of one or more anionic surfactants selected from the group consisting of alkyl sulfates and acyl methyl taurine salts is preferably from 0.5 to 5 mass %, more preferably from 1 to 3 mass %.

[22] The oral composition according to any one of aspects [1] to [21], wherein, in the case of the oral composition of the present invention being a liquid oral composition, a content of (H) water is preferably 50 mass % or more, more preferably 70 mass % or more, further preferably 80 mass % or more; and preferably 99 mass % or less, more preferably 95 mass or less, further preferably less than 90 mass %, and in the case of the oral composition of the present invention being a dentifrice composition, a content of ingredient (H) is preferably 10 mass % or more, more preferably 12 mass % or more; and preferably 60 mass % or less, more preferably 50 mass % or less.

[23] The oral composition according to any one of aspects [1] to [22], wherein the oral composition has a pH at 25° C. of preferably 5.8 or more; and 6.5 or less, preferably 6.2 or less when diluted with water to 30 mass %.

[24] The oral composition according to any one of aspects [1] to [23], wherein the viscosity at 25° C. is preferably 1,500 dPa·s or more, more preferably 1,800 dPa·s or more; and preferably 3,500 dPa·s or less, more preferably 3,000 dPa·s or less.

[25] The oral composition according to any one of aspects [1] to [24], wherein the oral composition is a dentifrice composition.

[26] The oral composition according to any one of aspects [1] to [24], wherein the oral composition is a liquid oral composition.

[27] Use of the oral composition according to any one of aspects [1] to [26] for removing stains in the interprismatic space on the tooth enamel surface layer.

[28] Use of the oral composition according to any one of aspects [1] to [26] for imparting a gloss to the teeth.

### EXAMPLES

The present invention will now be specifically described based on Examples. The content of each ingredient is represented by mass % unless otherwise specified in the Table.

### Examples 1 to 7, Comparative Examples 1 to 7

Liquid oral compositions were produced according to the formulations shown in Table 1. Using each of the obtained liquid oral compositions, each measurement and evaluation were carried out according to the following methods. The results are shown in Table 1.

<<Calcium Elution Amount Measurement of Low Crystalline HAp (Evaluation on Deposited Stain-Removing Effect in Interprismatic Space)>>

One g of hydroxyapatite (HAp) powder (HAP-100, Taihei Chemical Industrial Co., Ltd.) was taken in a styrene tube bottle (volume 120 mL), 50 mL of a slurry solution obtained by diluting each composition shown in Table 1 4 times with purified water was further added thereto and stirred for 15 minutes. After stirring, 1 mL of the solution was filtered using a 0.45 μm filter.

Subsequently, the eluted calcium ion was measured using Calcium E-test Wako (Wako Pure Chemical. Industries) to determine the calcium elution amount (ppm) of the low crystalline HAp and used as an indicator for evaluating the stain-removing effect in the interprismatic space.

The higher the value of elution amount is, it means that the stain-removing effect in the interprismatic space is good.

<<Calcium Elution Amount Measurement of High Crystalline HAp (Evaluation on Selectivity of Deposited Stain-Removing Effect in Interprismatic Space)>>

The calcium elution amount (ppm) of the high crystalline HAp was measured in the same manner as in the elution evaluation of low crystalline HAp except that HAP-200 (Taihei Chemical Industrial Co., Ltd.) was used in place of HAP-100 (Taihei Chemical Industrial Co., Ltd.) as the hydroxyapatite (HAp) powder and used as an indicator for evaluating the selectivity of stain-removing effect in the interprismatic space.

The lower the value of elution amount is, it means that the selectivity of deposited stain-removing effect in the interprismatic space is high and the occurrence of harm on the teeth surface is suppressed well.

<<Evaluation on Deposited Stain Adhesion-Suppressing Effect>>

An apatite flat plate (Pentax Corporation), sized 1 cm×1 cm, was allowed to stand in a styrene tube bottle (volume 50 mL), and 20 mL of each composition solution shown in Table 1 was added thereto and stirred for 10 minutes. After stirring, the apatite flat plate was washed with water and the surface gloss thereof was measured using a glossmeter NOVO CURVE (SANYO TRADING CO., LTD.). This measured value was defined as a gloss value before adhesion of deposited stain. Then, the apatite flat plate was again allowed to stand in the styrene tube bottle, and 40 mL of the deposited stain-producing solution was added thereto and stirred for 60 minutes. After stirring, the apatite flat plate was washed with water and the surface gloss was measured using the glossmeter. This measured value was defined as a gloss value after adhesion of deposited stain, with which a gloss reduction rate was calculated from the gloss value before adhesion of deposited stain and used as an indicator for the deposited stain adhesion-suppressing rate. The lower the gloss reduction rate is, it means that the adhesion of deposited stain is suppressed well.

TABLE 1

| | | | | | | | | | Comparative |
|---|---|---|---|---|---|---|---|---|---|
| | | Example 1 | Example 2 | Example 3 | Example 4 | Example 5 | Example 6 | Example 7 | Example 1 |
| | Potyethyleneglycol 600 | 5 | 5 | 5 | 5 | 5 | 5 | 5 | 5 |
| (E) | Sodium monofluorophosphate | 0.72 | 0.72 | 0.72 | 0.72 | 0.72 | 0.72 | 0.72 | 0.72 |
| | Cetyl pyridinium chloride | 0 | 0 | 0 | 0 | 0 | 0 | 0 | 0 |
| | Sorbitol (70%) | 31.5 | 31.5 | 31.5 | 31.5 | 31.5 | 31.5 | 31.5 | 31.5 |
| (A) | Phytic acid solution (50%) | 0.6 | 0.6 | 0.6 | 0.6 | 0.4 | 0.5 | 0.2 | 0.6 |
| (B) | Sodium tripolyphosphate | 0.5 | 1.0 | 0.3 | 0.8 | 0.2 | 1.0 | 0.2 | 0 |
| | Sodium pyrophosphate | 0 | 0 | 0 | 0 | 0 | 0 | 0 | 0 |
| (D) | Xylitol | 5 | 5 | 5 | 5 | 5 | 5 | 5 | 5 |
| | Sodium saccharin | 0 | 0 | 0 | 0 | 0 | 0 | 0 | 0 |
| | DL-malic acid | 0.1 | 0.1 | 0.1 | 0.1 | 0.1 | 0.1 | 0.1 | 0.1 |
| (H) | Purified water | 56.58 | 56.08 | 56.78 | 56.28 | 57.08 | 56.18 | 57.28 | 57.08 |
| Total | | 100 | 100 | 100 | 100 | 100 | 100 | 100 | 100 |
| (A) | Phytic acid effective amount | 0.3 | 0.3 | 0.3 | 0.3 | 0.2 | 0.25 | 0.1 | 0.3 |
| (H) | Water content | 66.33 | 65.83 | 66.53 | 66.03 | 66.73 | 65.88 | 66.83 | 66.83 |

| | | | | | | | | |
|---|---|---|---|---|---|---|---|---|
| (A) + (B) | 0.8 | 1.3 | 0.6 | 1.1 | 0.4 | 1.3 | 0.3 | 0.3 |
| (B)/(A) | 1.7 | 3.3 | 1.0 | 2.7 | 1.0 | 4.0 | 2.0 | 0.0 |
| pH | 6.0 | 6.0 | 6.0 | 6.0 | 6.0 | 6.0 | 6.0 | 6.0 |
| Ca elution amount of low crystalline | 9.5 | 10.5 | 8.3 | 9.8 | 7.4 | 10.7 | 7.2 | 2.5 |
| HAP [ppm] | | | | | | | | |
| Ca elution amount of high crystalline | 2.8 | 3.0 | 4.2 | 3.4 | 4.2 | 3.9 | 5.2 | 2.8 |
| HAP [ppm] | | | | | | | | |
| Deposited stain adhesion-suppressing | 5.8% | 9.1% | — | — | — | — | — | 13.4% |
| effect | | | | | | | | |

| | | | Comparative | Comparative | Comparative | Comparative | Comparative | Comparative |
|---|---|---|---|---|---|---|---|---|
| | | | Example 2 | Example 3 | Example 4 | Example 5 | Example 6 | Example 7 |
| | | Potyethyleneglycol 600 | 5 | 5 | 5 | 5 | 5 | 5 |
| | (E) | Sodium monofluorophosphate | 0.72 | 0.72 | 0.72 | 0.72 | 0.72 | 0.72 |
| | | Cetyl pyridinium chloride | 0 | 0 | 0 | 0 | 0 | 0 |
| | | Sorbitol (70%) | 31.5 | 31.5 | 31.5 | 31.5 | 31.5 | 31.5 |
| | (A) | Phytic acid solution (50%) | 1.6 | 0 | 0.6 | 0.6 | 1.2 | 2.0 |
| | (B) | Sodium tripolyphosphate | 0 | 1.0 | 0 | 1.0 | 1.0 | 1.0 |
| | | Sodium pyrophosphate | 0 | 0 | 1.0 | 0 | 0 | 0 |
| | (D) | Xylitol | 5 | 5 | 5 | 5 | 5 | 5 |
| | | Sodium saccharin | 0 | 0 | 0 | 0 | 0 | 0 |
| | | DL-malic acid | 0.1 | 0.1 | 0.1 | 0.1 | 0.1 | 0.1 |
| | (H) | Purified water | 56.08 | 56.68 | 56.08 | 56.08 | 55.48 | 54.68 |
| Total | | | 100 | 100 | 100 | 100 | 100 | 100 |
| | (A) | Phytic acid effective amount | 0.8 | 0 | 0.3 | 0.3 | 0.6 | 1 |
| | (H) | Water content | 66.33 | 66.13 | 65.83 | 65.83 | 65.53 | 65.13 |

| | | | | | | |
|---|---|---|---|---|---|---|
| (A) + (B) | 0.8 | 1.0 | 0.3 | 1.3 | 1.6 | 2.0 |
| (B)/(A) | 0.0 | — | 0.0 | 3.3 | 1.7 | 1.0 |
| pH | 6.0 | 6.0 | 6.0 | 8.0 | 6.0 | 6.0 |
| Ca elution amount of low crystalline | 4.9 | 12.7 | 6.0 | 4.4 | 6.6 | 5.0 |
| HAP [ppm] | | | | | | |
| Ca elution amount of high crystalline | 1.8 | 6.7 | 3.4 | 19 | 3.2 | 2.5 |
| HAP [ppm] | | | | | | |
| Deposited stain adhesion-suppressing | 12.5% | 19.5% | 16.0% | 18.2% | 22.1% | — |
| effect | | | | | | |

### Examples 8 to 10, Comparative Examples 8 to 10

Dentifrice compositions were produced according to the formulations shown in Table 2. Using the obtained dentifrice compositions, each measurement and evaluation were carried out in the same manner as in Example 1. The results are shown in Table 2.

TABLE 2

| | | | | Example | Comparative | Comparative | Comparative |
|---|---|---|---|---|---|---|---|
| | | Example 8 | Example 9 | 10 | Example 8 | Example 9 | Example 10 |
| | Polyethyleneglycol 600 | 5 | 5 | 5 | 5 | 5 | 5 |
| (E) | Sodium monofluorophosphate | 0.72 | 0.72 | 0.72 | 0.72 | 0.72 | 0.72 |
| | Cetyl pyridinium chloride | 0.01 | 0.01 | 0.01 | 0.01 | 0.01 | 0.01 |
| | Sorbit solution | 31.5 | 31.5 | 31.5 | 31.5 | 31.5 | 31.5 |
| (A) | Phytic acid solution (50%) | 0.6 | 0.6 | 0.5 | 0.6 | 0 | 1.2 |
| (B) | Sodium tripolyphosphate | 0.5 | 0.3 | 1 | 0 | 0.5 | 1.0 |
| | Sodium pyrophosphate | 0 | 0 | 0 | 0 | 0 | 0 |
| (D) | Xylitol | 5 | 5 | 5 | 5 | 5 | 5 |
| | Sodium saccharin | 0.13 | 0.13 | 0.13 | 0.13 | 0.13 | 0.13 |
| (F) | Xanthan gum | 0.2 | 0.2 | 0.2 | 0.2 | 0.2 | 0.2 |
| | Sodium carboxy methyl cellulose A*¹ (f) | 0.4 | 0.4 | 0.4 | 0.4 | 0.4 | 0.4 |
| | Sodium carboxy methyl cellulose B*² (f) | 0.7 | 0.7 | 0.7 | 0.7 | 0.7 | 0.7 |
| (G) | Anhydrous silicic acid A*³ | 7 | 7 | 7 | 7 | 7 | 7 |
| | Anhydrous silicic acid B*⁴ | 3 | 3 | 3 | 3 | 3 | 3 |
| | Anhydrous silicic acid C*⁵ | 2 | 2 | 2 | 2 | 2 | 2 |
| | Anhydrous silicic acid D*⁶ | 4 | 4 | 4 | 4 | 4 | 4 |
| | Granule*⁷ | 5 | 5 | 5 | 5 | 5 | 5 |
| | DL-malic acid | 0.1 | 0.1 | 0.1 | 0.1 | 0.1 | 0.1 |
| | Sodium lauryl sulfate | 1.4 | 1.4 | 1.4 | 1.4 | 1.4 | 1.4 |
| | Sodium lauroyl methyl taurate | 0.2 | 0.2 | 0.2 | 0.2 | 0.2 | 0.2 |
| | Titanium oxide | 0.3 | 0.3 | 0.3 | 0.3 | 0.3 | 0.3 |
| | Flavoring agent | 1.4 | 1.4 | 1.4 | 1.4 | 1.4 | 1.4 |
| (H) | Purified water | 30.84 | 31.04 | 30.44 | 31.34 | 31.44 | 29.74 |
| Total | | 100 | 100 | 100 | 100 | 100 | 100 |
| (A) | Phytic acid effective amount | 0.3 | 0.3 | 0.25 | 0.3 | 0 | 0.6 |
| (H) | Water content | 40.59 | 40.79 | 40.14 | 41.09 | 40.89 | 39.79 |

| | | | | | | |
|---|---|---|---|---|---|---|
| (A) + (B) | 0.8 | 0.6 | 1.3 | 0.3 | 0.5 | 1.6 |
| (B)/(A) | 1.7 | 1.0 | 4.0 | 0.0 | — | 1.7 |
| (B)/(D) | 0.10 | 0.06 | 0.20 | 0.00 | 0.10 | 0.20 |
| pH | 6.0 | 6.0 | 6.0 | 6.0 | 6.0 | 6.0 |
| Viscosity of dentifice [dPa · s] | 2600 | — | — | 2400 | — | — |
| Ca elution amount of low crystalline HAP [ppm] | 6.8 | 5.8 | 8.3 | 3.6 | 7.2 | 6.4 |
| Ca elution amount of high crystalline HAP [ppm] | 3.5 | 3.8 | 3.8 | 3.4 | 5.1 | 5.4 |

*¹SUNROSE F03HC, Etherification degree 0.85 to 0.95
*²SUNROSE F35SH, Etherification degree 1.00 to 1.15
*³Sylopure 25, Fuji Silysia Chemical Ltd.
*⁴Sylopure 39, Fuji Silysia Chemical Ltd.
*⁵Sorbosil AC44, Ineos Silica
*⁶Sorbosil AC77, ineos Silica
*⁷Contains colloidal silica 94%, cellulose 5%, and sodium hydroxide 1%

### Examples 11 to 16

Dentifrice compositions were produced according to the formulations shown in Table 3. Using the obtained dentifrice compositions, a friction feeling was evaluated according to the following method. The results are shown in Table 3 including the evaluation on the dentifrice composition obtained in Example 8.

<<Evaluation on Friction Feeling>>

Two subjects (1 male, 1 female) took 1 g of the dentifrice composition on a toothbrush, brushed as they like for about 2 minutes, and evaluated the friction feeling after rinsing the mouse according to the following criteria. The results of discussion by the subjects are shown in Table 3.

1: No friction sensed

2: Moderate friction sensed but negligible

3: Moderate friction sensed

4: Friction sensed

TABLE 3

| | | | Example | Example | Example | Example | Example | Example |
|---|---|---|---|---|---|---|---|---|
| | | Example 8 | 11 | 12 | 13 | 14 | 15 | 16 |
| | Polyethyleneglycol 600 | 5 | 5 | 5 | 5 | 5 | 5 | 5 |
| (E) | Sodium monofluorophosphate | 0.72 | 0.72 | 0.72 | 0.72 | 0.72 | 0.72 | 0.72 |
| | Cetyl pyridinium chloride | 0.01 | 0.01 | 0.01 | 0.01 | 0.01 | 0.01 | 0.01 |
| | Sorbit solution | 31.5 | 31.5 | 31.5 | 31.5 | 31.5 | 31.5 | 31.5 |
| (A) | Phytic acid solution (50%) | 0.6 | 0.6 | 0.6 | 0.6 | 0.6 | 0.6 | 1.0 |
| (B) | Sodium tripolyphosphate | 0.5 | 0.3 | 0.5 | 0.3 | 0.8 | 0.8 | 0.5 |
| | Sodium pyrophosphate | 0 | 0 | 0 | 0 | 0 | 0 | 0 |
| (D) | Xylitol | 5 | 5 | 10 | 10 | 5 | 10 | 5 |
| | Sodium saccharin | 0.13 | 0.13 | 0.13 | 0.13 | 0.13 | 0.13 | 0.13 |
| (F) | Xanthan gum | 0.2 | 0.2 | 0.2 | 0.2 | 0.2 | 0.2 | 0.2 |
| | Sodium carboxy methyl cellulose A*¹ (f) | 0.4 | 0.4 | 0.4 | 0.4 | 0.4 | 0.4 | 0.4 |
| | Sodium carboxy methyl cellulose B*² (f) | 0.7 | 0.7 | 0.7 | 0.7 | 0.7 | 0.7 | 0.7 |
| (G) | Anhydrous silicic acid A*³ | 7 | 7 | 7 | 7 | 7 | 7 | 7 |
| | Anhydrous silicic acid B*⁴ | 3 | 3 | 3 | 3 | 3 | 3 | 3 |
| | Anhydrous silicic acid C*⁵ | 2 | 2 | 2 | 2 | 2 | 2 | 2 |
| | Anhydrous silicic acid D*⁶ | 4 | 4 | 4 | 4 | 4 | 4 | 4 |
| | Granule*⁷ | 5 | 5 | 5 | 5 | 5 | 5 | 5 |
| | DL-malic acid | 0.1 | 0.1 | 0.1 | 0.1 | 0.1 | 0.1 | 0.1 |
| | Sodium lauryl sulfate | 1.4 | 1.4 | 1.4 | 1.4 | 1.4 | 1.4 | 1.4 |
| | Sodium lauroyl methyl taurate | 0.2 | 0.2 | 0.2 | 0.2 | 0.2 | 0.2 | 0.2 |
| | Titanium oxide | 0.3 | 0.3 | 0.3 | 0.3 | 0.3 | 0.3 | 0.3 |
| | Flavoring agent | 1.4 | 1.4 | 1.4 | 1.4 | 1.4 | 1.4 | 1.4 |
| (H) | Purified water | 30.84 | 31.04 | 25.84 | 26.04 | 30.54 | 25.54 | 30.44 |
| Total | | 100 | 100 | 100 | 100 | 100 | 100 | 100 |
| (A) | Phytic acid effective amount | 0.3 | 0.3 | 0.3 | 0.3 | 0.3 | 0.3 | 0.5 |
| (H) | Water content | 40.59 | 40.79 | 35.59 | 35.79 | 40.29 | 35.29 | 40.39 |

| | | | | | | | |
|---|---|---|---|---|---|---|---|
| (A) + (B) | 0.8 | 0.6 | 0.8 | 0.6 | 1.1 | 1.1 | 1.0 |
| (B)/(A) | 1.7 | 1.0 | 1.7 | 1.0 | 2.7 | 2.7 | 1.0 |
| (B)/(D) | 0.10 | 0.06 | 0.05 | 0.03 | 0.16 | 0.08 | 0.10 |
| pH | 6.0 | 6.0 | 6.0 | 6.0 | 6.0 | 6.0 | 6.0 |
| Viscosity of dentifrice [dPa · s] | 2600 | — | — | — | — | — | — |
| Friction feeling | 1 | 1 | 1 | 1 | 3 | 2 | 2 |

*¹SUNROSE F03HC, Etherification degree 0.85 to 0.95
*²SUNROSE F35SH, Etherification degree 1.00 to 1.15
*³Sylopure 25, Fuji Silysia Chemical Ltd.
*⁴Sylopure 39, Fuji Silysia Chemical Ltd.
*⁵Sorbosil AC44, Ineos Silica
*⁶Sorbosil AC77, Ineos Silica
*⁷Contains colloidal silica 94%, cellulose 5%, and sodium hydroxide 1%

<<Evaluation on Teeth Surface Gloss Using Dentifrice Composition of Example 8>>

The dentifrice composition obtained in Example 8 and the dentifrice composition of Comparative Example 11 having the formulation shown in Table 4 were used for 8 weeks and front teeth (4 teeth in total present between the upper lateral incisors on the labial side) were photographed on the first day of use, after 2 weeks of use, after 4 weeks of use, respectively. The brightness of the teeth was calculated from the images taken and used as an indicator for gloss. A toothbrush, a Clear Clean extra thick hair bundle toothbrush (Kao Corporation), distributed to the subjects was used. The Δ values of the brightness of the teeth after 2 weeks and 4 weeks of use were calculated, based on the brightness of the teeth on the first day of use. The higher an increase in the brightness is, it means that the ability to increase the gloss of the teeth is good.

Note that the teeth surface gloss can also be measured by a method using human extracted teeth and can be the indicator for evaluating the teeth surface gloss using the dentifrice composition of the present invention.

Specifically, human extracted teeth are divided into 2 parts in such a way as to separate into the buccal side and the lingual side, and fixed on an acrylic plate using a stickey wax. The fixed extracted teeth are photographed and the brightness of the teeth is calculated from the images taken and used as an indicator for the initial gloss. Then, the dentifrice composition of the present invention is taken onto a toothbrush to brush the extracted teeth. Subsequently, photographs are taken as in before brushing to calculate the brightness, which is used as an indicator for the gloss after brushing. The Δ values of the brightness after brushing were determined based on the initial brightness. In this case, the larger an increase in the brightness is, it means that the ability to increase the gloss of the teeth is good.

TABLE 4

| | | | Comparative |
|---|---|---|---|
| | | Example 8 | Example 11 |
| | Polyethyleneglycol 600 | 5 | 5 |
| (E) | Sodium monofluorophosphate | 0.72 | 0.72 |
| | Cetyl pyridinium chloride | 0.01 | 0.01 |
| | Sorbit solution | 31.5 | 31.5 |
| (A) | Phytic acid solution (50%) | 0.6 | 0 |
| (B) | Sodium tripolyphosphate | 0.5 | 0 |
| | Sodium pyrophosphate | 0 | 0 |
| (D) | Xylitol | 5 | 5 |
| | Sodium saccharin | 0.13 | 0.13 |
| (F) | Xanthan gum | 0.2 | 0.2 |
| | Sodium carboxy methyl cellulose | 0.4 | 0.4 |
| | A*¹ (f) | | |
| | Sodium carboxy methyl cellulose | 0.7 | 0.7 |
| | B*² (f) | | |
| (G) | Anhydrous silicic acid A*³ | 7 | 7 |
| | Anhydrous silicic acid B*⁴ | 3 | 3 |
| | Anhydrous silicic acid C*⁵ | 2 | 2 |
| | Anhydrous silicic acid D*⁶ | 4 | 4 |
| | Granule*⁷ | 5 | 5 |
| | DL-malic acid | 0.1 | 0.1 |
| | Sodium lauryl sulfate | 1.4 | 1.4 |
| | Sodium lauroyl methyl taurate | 0.2 | 0.2 |
| | Titanium oxide | 0.3 | 0.3 |
| | Flavoring agent | 1.4 | 1.4 |
| (H) | Purified water | 30.84 | 31.94 |
| Total | | 100 | 100 |
| (A) | Phytic acid effective amount | 0.3 | 0 |
| (H) | Water content | 40.59 | 41.39 |
| (A) + (B) | | 0.8 | 0.0 |
| (B)/(A) | | 1.7 | — |
| (B)/(D) | | 0.10 | 0.00 |
| pH | | 6.0 | 6.0 |
| Viscosity of dentifrice [dPa · s] | | 2600 | — |
| Teeth | 0 W Brightness | 160.0 | 148.0 |
| surface | 2 W Brightness | 185.5 | 167.9 |
| gloss | 4 W Brightness | 194.0 | 177.9 |
| | 2 W Brightness [symbol] | 25.5 | 19.9 |
| | 4 W Brightness [symbol] | 34.0 | 29.9 |

*¹SUNROSE F03HC, Etherification degree 0.85 to 0.95
*²SUNROSE F35SH, Etherification degree 1.00 to 1.15
*³Sylopure 25, Fuji Silysia Chemical Ltd.
*⁴Sylopure 39, Fuji Silysia Chemical Ltd.
*⁵Sorbosil AC44, Ineos Silica
*⁶Sorbosil AC77, Ineos Silica
*⁷Contains colloidal silica 94%, cellulose 5%, and sodium hydroxide 1%

Dentifrice having the following formulation can be produced by a routine method as the oral composition of the present invention.

[Formulation Example 1 Dentifrice]

| | | |
|---|---|---|
| Polyethyleneglycol 600 | 5 | mass % |
| Sodium monofluorophosphate | 1.1 | mass % |
| Cetyl pyridinium chloride | 0.01 | mass % |
| Sorbitol (70%) | 31.5 | mass % |
| Phytic acid solution (50%) | 0.6 | mass % |
| Sodium tripolyphosphate | 0.5 | mass % |
| Xylitol | 5 | mass % |
| Sodium saccharin | 0.13 | mass % |
| Xanthan gum | 0.2 | mass % |
| Carboxy methyl cellulose A*¹ | 0.4 | mass % |
| Carboxy methyl cellulose B*² | 0.7 | mass % |
| Anhydrous silicic acid A*³ | 7 | mass % |
| Anhydrous silicic acid B*⁴ | 3 | mass % |
| Anhydrous silicic acid C*⁵ | 2 | mass % |
| Anhydrous silicic acid D*⁶ | 4 | mass % |
| Granule*⁷ | 5 | mass % |
| DL-malic acid | 0.1 | mass % |
| Sodium lauryl sulfate | 1.4 | mass % |
| Lauroyl methyl taurine | 0.2 | mass % |
| Titanium oxide | 0.3 | mass % |
| Flavoring agent | 1.4 | mass % |
| Purified water | 30.46 | mass % |
| Total | 100 | mass % |

*¹ to ⁷Same as Table 2

1. An oral composition comprising the following ingredients (A) and (B):
(A) phytic acid or a salt thereof, and
(B) tripolyphosphoric acid or a salt thereof,
wherein a total content of ingredient (A) in terms of phytic acid and ingredient (B) in terms of acid is 0.02 mass % or more and 1.4 mass % or less, and a mass ratio of ingredient (B) in terms of acid to ingredient (A) in terms of phytic acid, (B)/(A), is 0.8 or more and 5 or less,
the oral composition does not comprise one or more polyvalent metals selected from the group consisting of (C) copper, iron, calcium, magnesium, aluminum, zinc, and tin, or a salt thereof, or comprise ingredient (C) in a molar amount of less than 0.1 times that of ingredient (A) in terms of phytic acid, and
the oral composition has a pH at 25° C. of 5.5 or more and 6.5 or less when diluted with water to 30 mass %.

2. The oral composition according to claim 1, wherein a content of ingredient (A) is from 0.01 to 0.7 mass %.

3. The oral composition according to claim 1, wherein the oral composition further comprises (D) xylitol.

4. The oral composition according to claim 3, wherein a mass ratio of the content of ingredient (B) to the content of ingredient (D), (B)/(D), is 0.01 or more and 0.04 or less.

5. The oral composition according to claim 3, wherein a mass ratio of the total content of ingredient (A) in terms of phytic acid and ingredient (B) in terms of acid to the content of ingredient (D), {(A)+(B)}/(D), is 0.02 or more and 0.35 or less.

6. The oral composition according to claim 1, wherein the oral composition further comprises (E) monofluorophosphoric acid or an alkali metal salt thereof.

7. The oral composition according to claim 1, wherein the oral composition further comprises (F) a binder containing a cellulose binder (f).

8. The oral composition according to claim 1, wherein the oral composition further comprises 1 mass % or more and 15 mass % or less of (G) thickening silica having an oil absorption of 200 mL/100 g or more and 400 mL/100 g or less.

9. The oral composition according to claim 1, wherein the oral composition further comprises 10 mass % or more and 99 mass % or less of (H) water.

10. The oral composition according to claim 1, wherein a viscosity at 25° C. is 1,500 dPa·s or more and 3,500 dPa·s or less.

11. The oral composition according to claim 7, wherein the content of ingredient (F) other than ingredient (f) is 0.01 mass % or more and 1 mass % or less.

12. The oral composition according to claim 7, wherein the oral composition comprises carboxy methyl cellulose or a salt thereof as the cellulose binder (f).

13. The oral composition according to claim 1, wherein the oral composition further comprises granules containing a water-insoluble inorganic binder.

14. The oral composition according to claim 13, wherein the content of the water-insoluble inorganic binder in the granules is from 10 to 45 mass %.

15. The oral composition according to claim 13, wherein the content of the granules containing the water-soluble inorganic binder is 1 mass % or more and 10 mass % or less.

16. The oral composition according to claim 1, wherein the oral composition has a pH at 25° C. of 5.8 or more and 6.5 or less when diluted with water to 30 mass %.

17. The oral composition according to claim 1, wherein the oral composition is a dentifrice composition.

18. The oral composition according to claim 1, wherein the oral composition is a liquid oral composition.

19. The oral composition according to claim 1,
wherein the oral composition is a liquid oral composition, and a content of one or more anionic surfactants selected from the group consisting of alkyl sulfates and acyl methyl taurine salts is from 0.5 to 5 mass %; or
wherein the oral composition is a dentifrice composition, and a content of anionic surfactant selected from the group consisting of alkyl sulfates and acyl methyl taurine salts is from 0.5 to 5 mass %.

20. A method for removing stains in the interprismatic space on the tooth enamel surface layer, comprising applying the composition according to claim 1 to the oral cavity.

21. A method for imparting a gloss to the teeth, comprising applying the composition according to claim 1 to the oral cavity.
